(19) United States
(12) Patent Application Publication
(10) Pub. No.: US 2015/0140135 A1
(43) Pub. Date: May 21, 2015

(54) METHOD OF USING DIETARY SUPPLEMENTS COMPRISING CINNAMON EXTRACTS TO PROMOTE WEIGHT LOSS

(71) Applicants: Peter J. Miller, Broomfield, CO (US); Timothy Romero, Columbia, TN (US)

(72) Inventors: Peter J. Miller, Broomfield, CO (US); Timothy Romero, Columbia, TN (US)

(73) Assignee: IN Ingredients, Inc., Columbia, TN (US)

(21) Appl. No.: 14/610,311

(22) Filed: Jan. 30, 2015

Related U.S. Application Data
(63) Continuation of application No. 11/182,701, filed on Jul. 15, 2005.
(60) Provisional application No. 60/521,885, filed on Jul. 16, 2004.

(51) Int. Cl. A61K 36/54; A61K 36/74; A23L 1/30; A61K 31/685; A61K 36/24; A61K 31/522; A61K 36/82; A61K 36/575

(52) U.S. Cl. CPC A61K 36/54; A61K 36/82; A61K 36/74; A61K 36/575; A61K 31/685; A61K 36/24; A61K 31/522; A23L 1/3002; A61K 2236/331; A61K 2236/333; A23V 2002/00
USPC 424/729; 424/739

(57) ABSTRACT

A method for promoting weight loss and/or lowering blood glucose comprises an active material which may be cinnamon, an extract of cinnamon, or a derivative of a cinnamon extract. The active material may be utilized in combination with further active ingredients.

### RELATED APPLICATION

This application claims priority of U.S. Provisional Patent Application Ser. No. 60/521,885 filed Jul. 16, 2004, entitled “Dietary Supplements Containing Extracts of Cinnamon and Methods of Using Same to Promote Weight Loss,” which is incorporated herein by reference.

### FIELD OF THE INVENTION

The present invention is directed to dietary supplements comprising cinnamon, or extracts thereof or derivatives of the extracts thereof, and to methods of using these dietary supplements to promote weight loss, both in humans and animals.

### BACKGROUND

Obesity and Type II diabetes are quickly becoming an epidemic in the United States. The increased incidence of these conditions has been attributed to diets characterized by high fat intake and repeated ingestion of refined foods and sugars, coupled with low fiber and vegetable intake. Diet, along with the natural aging process, causes deterioration in the way in which the body metabolizes blood glucose. When the body cannot properly metabolize blood glucose, a tendency to store glucose as fat typically occurs. This is one reason levels of body fat increase with age. There is also a known link with these conditions to a variety of ailments including heart disease and hypertension. Similarly, there is a known link between insulin resistance and increased visceral adiposity. Therefore, when glucose regulation is out of balance, a greater propensity for adiposity exists.

It has long been known that natural and/or synthetic substances may aid in controlling blood glucose. Such substances act by a variety of mechanisms. For example, some substances act by mimicking the effects of endogenous insulin and are therefore capable of replacing endogenous insulin. Such substances include synthetic insulin injections such as those which are routinely prescribed to individuals with Type I diabetes. Other commonly prescribed substances known to mimic the effects of insulin include the naturally occurring compounds taurine, 4-hydroxyisoleucine, arginine, and vanadium. Although these compounds have been shown to work as insulin mimetics by acting in the body to decrease serum blood glucose levels, they have not been successfully developed into viable treatments for disorders of glucose metabolism.

Still other substances act directly to increase what is termed insulin sensitivity or glucose tolerance. Glucose intolerance forces the body to generate additional insulin in an effort to lower blood glucose. This causes stress on the beta-cells of the pancreas and is thought to be a key contributor to Type II diabetes. In a state of glucose intolerance, the body mechanism for disposing of blood glucose is not functioning at its optimum level and therefore the system is inefficient. Substances which increase insulin sensitivity or glucose tolerance by assisting the body in returning to optimal levels of blood glucose include alpha-lipoic acid, pinitol and myo-inositol. These substances cannot entirely replace the function of endogenous insulin, but work at the receptor level alongside endogenous insulin to increase insulin sensitivity or glucose tolerance. Here, the action is exerted directly on the Glut-4 receptor of the cell to trigger the cascade normally caused by insulin that allows for the reduction in blood sugar via the transport of nutrients into the cell.

In the past, chromium was thought to aid in weight loss by controlling blood glucose and preventing the deposition of fatty acids. However, its actions were greatly limited and its claims never came to fruition. Cinnamon, known for its high concentration of chromium, has also been used for the control of blood glucose. However, researchers have demonstrated that cinnamon's effects are not from chromium, but rather from a different class of compounds. One study by Kahn et al. compared the chromium levels of foods and spices including cinnamon, and failed to find a correlation between chromium level and the level of insulin potentiation. (Biological Trace Element Research, 1990; 24:183-188). A meta-analysis by Althuis et al. showed no association between chromium and glucose or insulin concentrations. (Am. J. Clin. Nutr., 2002; 76:148-55). A study by Broadhurst et al. has demonstrated that cinnamon is a strong potentiator of insulin in comparison to various other herbs and spices. (J. Agric. Food Chem., 2000; 48:849-852).

One particular extract of cinnamon, methyl hydroxy chalcone polymer (MHCP), shows promising data in the area of glucose control. A recent study compared the effect of MHCP in 3T3-L1 adipocytes to that of insulin. (Jarvill-Taylor et al., J. Am. College Nutr., 2001; 20:327-336). The results from that study support the theory that MHCP triggers the insulin cascade and subsequent transport of nutrients. The study also demonstrated that MHCP treatment stimulated glucose uptake and glycogen synthesis to a similar level as insulin. The study further demonstrated that treatment with endogenous insulin and MHCP resulted in a synergistic effect. Due to these conclusions it is suggested that MHCP may prove to be a very valuable tool in the fight against diabetes, where insulin is present.

In addition to benefiting Type II diabetics, cinnamon may benefit individuals with impaired glucose tolerance (i.e., pre-diabetics). Further, cinnamon has been shown to possess antioxidant activities related to lipid peroxidation. (Mancini-Filho et al., Bollettino Chimico Farmaceutico, 1998; 37:443-47). Cinnamon can be used as a food antioxidant and to enhance food palatability.

There exists a need in the art for a safe, effective and viable method promoting weight loss. Further, there exists a need in the art for a material which can be incorporated into a pharmaceutical formulation, a dietary supplement or a food product, whose administration at normal physiological concentrations would promote weight loss.

### BRIEF DESCRIPTION OF THE INVENTION

Disclosed herein is: (a) a dietary supplement comprising cinnamon, or an extract thereof or a derivative of the extract thereof and (b) methods of losing weight and reducing body fat comprising administration of said dietary supplement.

### DETAILED DESCRIPTION

The body fat reduction and weight loss dietary supplements of the invention comprise cinnamon, or an extract thereof or a derivative of the extract thereof. The materials of the present invention are effective when administered orally; however, intravenous, intramuscular or transdermal delivery of these materials may also be employed. The active materials of the present invention may be incorporated into pharmaceutical preparations. They may also be used in dietary supplements, and may also be added directly to food products.

Cinnamon is one of the world's most popular spices. Cinnamon contains over one hundred different chalcones within it. Chalcones are a type of polyphenol or flavonoid. These chalcones or polymers may be extracted from cinnamon and isolated, and, optionally, derivatized. One class of polyphenols which can be extracted from cinnamon is the phytochemical Type A polyphenols. In a specific embodiment of the invention, the dietary supplement includes Type A Polyphenols. One particular cinnamon derived material having utility in this invention is methyl hydroxy chalcone polymer (MCHP).

The isolation of phytochemicals from cinnamon according to one method having utility in this invention follows the general process of aqueous extraction followed by centrifugation to remove non-soluble compounds. In one preparation method, Type A polyphenols are extracted from cinnamon using the following process: 5 g cinnamon and 100 ml 0.1 N acetic acid are combined and autoclaved for 15 minutes. The resultant mixture is cooled, then centrifuged and the precipitate discarded. Four volumes of ethanol/0.1 N acetic acid are added to the supernatant and the mixture is stored overnight at 4 C°. The mixture is screened through a filter and then introduced onto an LH-20 column and washed with 600 ml ethanol/0.1 N acetic acid. The desired fraction is then eluted with a 1:1 mixture of acetonitrile and 0.2 N acetic acid. The eluant is then concentrated and introduced onto a HPLC column at 275 nm.

Another method for producing an extract which may be used in the practice of the present invention is as follows:
1. Choose clean cinnamon bark about 50 g (from Indonesia), grind into small particles or powder. Regulate the temperature of the grinder.
2. Weigh about 20 g ground cinnamon powder into a suitable flask, mix with 1000 mL distilled water. Leave at room temperature for about 0.5 hour. The amount of water added to the raw material for extraction is in a weight ratio of about 1:50. In general, the range can be from 1 to 1:200. If the ratio is too low (1:20), the extraction liquid will be very thick, not easy to filter, and the extraction efficiency is also lower. If the ratio is too high (1:200), the volume of extraction solution will be much greater resulting in an increase of drying time.
3. Heat and stir the cinnamon liquid on a magnetic heat stirrer. The bioactive polymers in the cinnamon are relatively heat sensitive. The temperature and extraction time is crucial to the concentration of the bioactive polymers. The extraction process should be no longer than one hour. As outlined below:
   a) 15-20 minutes bring to boil while stirring constantly.
   b) 20 minutes boiling and stir constantly.
   c) 20-30 minutes simmer stirring constantly after turning down the heat (temperature is about 80-95° C.).
   It is better to control the boiling time about 20-25 minutes. Move the flask from the heater; after it cools down, store at 4° C. for overnight.
4. Filter the solution through a filter paper to remove any solid debris. If the solution is too thick to go through the filter paper, centrifuge can be used for separation. The supernatant can be separated first with a pipette, then filter the rest of the solution with the medium speed filter paper. Usually the debris settles to the bottom of the flask. It is not necessary to filter this debris.
5. If the raw material and the water ratio are low, a second extraction is needed. Add 200 mL distilled water into the residual debris, mix and heat the solution for 30 minutes at 90° C. Filter it and mix the first and second extraction solutions together. Note: The sample and water ratio, heat time, volume of water in second extraction may vary depending on the amount of the raw material used for extraction.
6. Pour the extraction solution into a nonstick tray, and dry it in an oven at 80-90° C. Do not overheat or boil the solution. If vacuum spray dry equipment is available, it is acceptable to use.
7. Collect the dry cinnamon powder, weigh it. Calculate the extraction ratio.

%=w/20×100%
w: the weight of the cinnamon extract powder.

In one experimental series, an extract was prepared according to the foregoing procedure. Weigh 100 mg extract powder into 100 mL volumetric flask, dilute with water to 100 m, sonicate the solution for 30-45 minutes. Filter the solution through 0.45 um PTFE syringe, determine the polymers according to the INT procedure. The concentration of the sample was approximately 5.17 mg/ml. It is also very important to note that the concentrations of the polymers change with the temperature and extraction time.

Samples were extracted at 50-60° C. for about one hour, polymers eluting at 17 and 21 minutes seemed to have reasonable concentrations. After increasing the temperature to 75-82° C. for one hour, the peaks eluting at 17 and 21 minutes decreased by about 2-3%. There are an additional two relatively small peaks that seemed to surface during this extraction. They eluted at 28.5 minutes and 33.5 minutes, respectively. After increasing the heat to 85-90° C. for an additional hour, the peaks eluting at 17 and 21 minutes decreased about 7-9%. The peaks at 28.5 and 33.5 minutes increase significantly. Finally, the temperature was increased to 95-100° C. for 20 minutes, and heat was then reduced to 85-95° C. for an additional 40 minutes. The results in peaks eluting 17 and 21 minutes seemed to decrease about 15-20%.

The peaks eluting at 28.5 and 33.5 minutes increase more than double from the previous. According to these results, the polyphenols at 17 and 21 minutes are believed to convert to isomers at 28.5 and 33.5 minutes respectively. These results suggest that the extraction at 100° C. seemed to yield the highest concentration of polymers.

An additional experiment was conducted on the extract at 100° C. to verify stability. Samples were extracted at 95-100° C. for about one hour, polymers eluting at 17 and 21 minutes seemed to have reasonable concentrations. The peaks eluting at 17 and 21 minutes decrease as the temperature increases in the first 2-3 hours. After 3 hours, the peaks eluting at 17 and 21 minutes did not change significantly. The peak area at 28.5 and 33.5 minutes increased temperature in the first 2-3 hours. After 3 hours the peaks eluting at 28.5 and 33.5 minutes did not change significantly. These results suggest after 3 hours, these polymers seem to stabilize.

Not only is it important to note that the time and temperature play a key factor in sustaining higher concentrations of these key actives, additionally the species of choice can have a dramatic impact on the levels of these Type-A polymers. The following species appear to provide the highest level of active Type-A polymers: Cinnamomum Burmannii (Nees) Blume—Microbial Identification I (MIDI) class; Korintji Cassia. Concentrations of the bioactive polymers appear to be much higher in Indonesian cinnamon versus several other samples.

U.S. Pat. No. 6,200,569 discloses the preparation of particular botanical extracts having utility in the treatment of diabetes and similar conditions. Specifically disclosed therein are some specific methods for preparing cinnamon extracts and derivatives of the type having utility in the practice of the present invention. The entire disclosure of the U.S. Pat. No. 6,200,569 patent is incorporated by reference herein. One of skill in the art could, by reference to the incorporated patent, prepare cinnamon-based materials having utility in the present invention.

Typical formulations used in the practice of the present invention for promoting weight loss and/or reduction of body fat generally include 1-10,000 mg of the cinnamon material. In specific embodiments, levels of cinnamon derived materials in the range of 100-500 mg are utilized. The foregoing dosages are based upon the weights of the raw extract powder. The active Type-A polymer fraction of such powders is approximately 1%, so dosages based solely on the active material will be in the approximate range of 100 mcg to 1000 mcg.

Formulations of the present invention may simply comprise a pharmaceutical preparation of a liquid, encapsulated liquid, or solid form of the material. In some instances, the active ingredient of the present invention may be disposed in a food product such as a biscuit, energy bar, or the like. However, in particular instances, the active cinnamon-based material of the present invention is included as a part of a dietary supplement or nutraceutical which may include further active ingredients, as well as inactive ingredients such as flavoring agents, coloring agents, carriers, fillers, and the like. In specific formulations, the active, cinnamon-based material is utilized at a level of approximately 100-500 mg, and this dosage is typically consumed on a daily basis.

One specific formulation for promoting glucose control based weight loss includes a cinnamon-based extract of the type disclosed in the U.S. Pat. No. 6,200,569 patent, said extract being referred to herein as Cinnulin PF. This specific formulation comprises:

| | | |
|---|---|---|
| Cinnulin PF: | 250 | mg |
| Chromium picolinate: | 100 | mcg |
| Green tea 45% EGCG: | 250 | mg |
| Gymnema sylvestre: | 100 | mg |
| Alpha-lipoic acid (r): | 100 | mg |
| Green coffee (chlorogenic acid): | 100 | mg |

Another formulation in accord with the present invention for thermogenic based glucose and/or weight control comprises:

| | | |
|---|---|---|
| 7-keto DHEA: | 00 | mg |
| Cinnulin PF: | 250 | mg |
| Evodiamine: | 25 | mg |

| | | |
|---|---|---|
| Oolong tea: | 100 | mg |
| Green tea 45% EGCG: | 300 | mg |
| Caffeine anhydrous: | 200 | mg |
| Yohimbine: | 3 | mg |

A formulation of the present invention for control of weight and/or glucose which functions as an appetite control agent comprises:

| | | |
|---|---|---|
| Hoodia gordonii: | 100 | mg |
| Cinnulin PF: | 250 | mg |
| Glucommanan (konjac): | 500 | mg |
| Chromium picolinate: | 100 | mcg |

A formulation which promotes weight loss and/or moderates glucose and further operates to lower cortisol comprises:

| | | |
|---|---|---|
| Magnolia officianalis: | 100 | mg |
| Phosphatidyl serine: | 300 | mg |
| Cinnulin PF: | 250 | mg |

Yet another formulation of the present invention which further operates to block carbohydrates includes Cinnulin PF in an amount of typically 250 mg together with a carbohydrate blocking material such as Phase 2 or a starch blocking substance in the amount of 500 mg.

The efficacy and safety of the method of the present invention was evaluated in a twelve-week, double-blind, placebo-controlled, randomized group study conducted by the Ohio Research Group in Wadsworth, Ohio. Subjects were randomized and received either the Cinnulin PF paste composition or a placebo supplement for a twelve-week period. Minimal steps were taken to influence subjects' lifestyle changes with regard to diet or exercise. The subjects comprised 24 persons having fasting glucose levels between 100 mg/dl and 139 mg/dl, and ranging in age between 23 and 64 years. The subjects' weight, percent body fat, fat mass, fasting glucose, fructosamine, blood pressure and pulse were measured at zero days, six weeks and twelve weeks post-randomization. The study showed that subjects receiving the Cinnulin PF treatment experienced on the average 2.1% reduction in body fat, compared to the placebo group of subjects who gained 0.9% body fat. In addition, the subjects receiving the Cinnulin treatment experienced an average 9.18 mg/dl reduction in blood glucose compared with a 1.1 mg/dl increase in blood glucose for the placebo group. There were no significant differences observed in the two groups with respect to diastolic blood pressure, pulse, or the occurrence of any adverse event. Based upon the twelve-week trial, it was demonstrated that the subjects consuming the Cinnulin PF supplement experienced a significant reduction in body fat percentage without an increase in blood pressure, pulse or the rate of any adverse events. These benefits were achieved in the absence of any major lifestyle treatment to change dietary or exercise behavior.

In a typical regimen, the foregoing materials are taken orally between one and three times daily; although, other routes of administration may be utilized as noted above. In other embodiments, the various ingredients listed above may be utilized in other combinations and/or at other dosage levels. Also, it should be noted that the extracts of the present invention may be utilized in the form of derivatives. For example, the extracts may be bonded, chemically or physically, to other species and moieties such as synthetic polymers, liposomes, small organic molecules, chitin, chitosan, other biopolymers and the like. In view of the teaching presented herein, still further combinations will be readily apparent to those of skill in the art.

The foregoing discussion and description is illustrative of specific embodiments of the present invention, but is not meant to be a limitation upon the practice thereof. Further modifications and variations of the invention will be readily apparent to those of skill in the art. It is the following claims, including all equivalents, which define the scope of the invention.

1-16. (canceled)

17. A method for promoting weight loss in an animal in need thereof, said method comprising:
preparing an extract of cinnamon (Cinnamomum sp.), said step of preparing comprising
(i) heating cinnamon in an aqueous or organic extraction solvent;
(ii) separating out a liquid portion; and
(iii) further isolating type-A polyphenols from said liquid portion to form said extract of cinnamon; and
orally administering on a daily basis to said animal an amount between 1 and 10,000 mg by weight of said extract of cinnamon to promote weight loss in said animal, wherein said extract consists essentially of one or more type-A polyphenols.

18. The method of claim 17, wherein said cinnamon is Indonesian cinnamon.

19. The method of claim 17, wherein said one or more type-A polyphenols is methylhydroxychalcone polymer.

20. The method of claim 17, wherein said step of administering comprises administering between 100 milligrams and 500 milligrams of said extract of cinnamon.

21. The method of claim 17, wherein said extract of cinnamon is further combined with a material selected from the group consisting of: chromium picolinate, green tea extract, gymnema sylvestre, alpha-lipoic acid, green coffee extract, 7-keto DHEA, evodiamine, oolong tea, caffeine, yohimbine, hoodia gordonii, glucomannan, magnolia officianalis, phosphatidyl serine, a carbohydrate blocking agent, and combinations thereof.

22. The method of claim 17, wherein said animal is a mammal.

23. The method of claim 17, wherein said animal is a human.

24. The method of claim 17, wherein said composition is disposed in a food product.

25. The method of claim 17, wherein said extract of cinnamon is disposed in a nutritional supplement.

26. The method of claim 17 wherein said extraction solvent consists of water.

27. The method of claim 17 wherein said extraction solvent comprises ethanol/0.1 N acetic acid.

28. The method of claim 17 wherein said step of isolating is by chromatography.

29. A method for promoting weight loss in an animal in need thereof, said method comprising:
preparing an aqueous extract of cinnamon by extracting cinnamon in a solvent consisting essentially of water at a temperature of 50-60 degrees Celsius for one hour; and
administering to said animal, said aqueous extract of cinnamon wherein said extract comprises type-A polyphenols.

30. A method of preparing an extract of cinnamon comprising:
(i) heating cinnamon in an aqueous or organic extraction solvent;
(ii) separating out a liquid portion; and
(iii) further isolating one or more type-A polyphenols from said liquid portion to form said extract of cinnamon.

31. The method of claim 30 wherein said cinnamon is Indonesian cinnamon.

32. The method of claim 30, wherein said one or more type-A polyphenols is methylhydroxychalcone polymer.

33. The method of claim 30 wherein said extraction solvent consists of water.

34. The method of claim 30 wherein said extraction solvent comprises ethanol/0.1 N acetic acid.

35. The method of claim 30 wherein said step of isolating is by chromatography.

36. The method of claim 30, further comprising disposing said extract of cinnamon in a nutritional supplement.
